(19) United States
(12) Patent Application Publication
(10) Pub. No.: US 2022/0404339 A1
(43) Pub. Date: Dec. 22, 2022

(54) PROLONGED FUNCTION OF LIVER ORGANOIDS BY 3D COCULTURING OF HEPATIC LINAGE CELLS AS AN IN VITRO MODEL FOR THE STUDY OF LIVER DISEASE

(71) Applicant: THE TRUSTEES OF INDIANA UNIVERSITY, Indianapolis, IN (US)

(72) Inventor: Burcin Ekser, Indianapolis, IN (US)

(21) Appl. No.: 17/761,121

(22) PCT Filed: Sep. 17, 2020
(86) PCT No.: PCT/US2020/051216

§ 371 Date: Mar. 16, 2022

Related U.S. Application Data
(60) Provisional application No. 62/901,292, filed on Sep. 17, 2019.

(51) Int. Cl. G01N 33/50; C12N 5/071

(52) U.S. Cl. CPC G01N 33/5082; C12N 5/0671; G01N 33/5023; C12N 2501/15; C12N 2501/42; C12N 2501/415

(57) ABSTRACT

Disclosed is a spheroid liver organoid comprising hepatic lineage cells such as human hepatocytes, hepatic stellate cells, and liver sinusoidal endothelial cells. Also provided are methods of using spheroid liver organoids for applications related to drug screening and toxicity screening. In particular, spheroid liver organoids are useful for high-throughput screens to identify compounds having efficacy for treating liver disease.

### CROSS-REFERENCE TO RELATED APPLICATIONS

This application claims priority to U.S. Provisional Application No. 62/901,292, filed Sep. 17, 2019, which is hereby incorporated by reference herein in its entirety.

### GOVERNMENT SUPPORT CLAUSE

This invention was made with government support under TR002529 awarded by the National Institutes of Health. The government has certain rights in the invention.

### BACKGROUND

The global liver disease therapeutics market totaled $24.5 billion in 2014, with the U.S. contributing $9.1 billion, and is anticipating a global market of $33.8 billion by 2019. Driven by the obesity and diabetes epidemics, the NAFLD/NASH diseases guarantee an enormous pool of patients for decades, making it a prime target for deals for promising therapies and its consequences such as advanced fibrosis and liver-destroying cirrhosis.

Cirrhosis is a major source of morbidity/mortality and frequently occurs following chronic insult to trigger wound healing responses in the liver. Liver transplantation is the mainstay of current therapy for end stage liver failure and cirrhosis. To date, no in vitro liver model has been established that can solidly maintain long-term physiological function. Therefore, there is a critical need to develop novel cellular approaches for drug toxicity screening and liver disease models.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, wherein:

FIGS. 1A-1D show human liver organoid formation by 3D co-culturing of hepatic linage cells. FIG. 1A shows images of isolated Human Hepatocytes (HC), Hepatic Stellate Cells (HSC) and Liver Sinusoidal Endothelial Cells (LSEC) under phase contrast microscopy. FIG. 1B shows the schematic of 3D liver spheroids formation protocol including plating and maintaining cell conditions with HC maintenance medium (“HMM”). HMM=hepatocyte maintenance medium, CM=Change of culture medium. FIG. 1C shows the progression of HC, HC:HSC and HC:HSC:LEC spheroid formation on days 3, 5 and 8. Shown in FIG. 1D are (Top) co-immunofluorescent staining (co-IF) results for HC expression of albumin and HSC expression of Crbp 1 in liver spheroids. (Bottom) Co-IF results for HC expression of HNF4a, and LSEC expression of CD31.

FIGS. 2A-2E illustrate the chemical conditions (1C, 2C, 3C, 5C) for prolonged liver organoids culturing. FIG. 2A lists of chemical compounds that were used in culturing liver spheroids. FIG. 2B demonstrates spheroid culturing with 5C, 3C, 2C, and 1C medium. FIG. 2C depicts images of spheroid formations in each of the spheroid type and chemical conditions. AM: Adding medium; CM: Changing medium. FIG. 2D shows spheroid diameter and roundness in different chemical conditions and cell types were characterized by Regenova Bioprinter (Cyfuse Biomedical, Japan). FIG. 2E shows (top): qRT-PCR showing that the expression of cholangiocyte marker, CK19, in indicated spheroid type and medium conditions; (bottom), co-immunofluorescent (co-IF) staining showing the expression CK18 and CK19 in HC:HSC and HC:HSC:LEC spheroids plated in 2C and 3C media.

FIGS. 3A-3C demonstrate that the 3C medium preserved the hepatic function of cultured human liver organoids. FIG. 3A shows qRT-PCR analysis for Albumin (ALB) and CYP2B6 expression in HC:HSC:LEC spheroid and indicated medium after 7 days of culture in indicated medium, including chemical conditioned medium (total 14 days). FIG. 3B Co-IF illustrates expression of ALB and CK-7. FIG. 3C Co-IF staining of Sox9 and Pan-Cyotokeratin (Pan-CK) in HC:HSC and HC:HSC:LEC spheroids conditions after 14 days of culture in chemical conditioned medium (total 14 days).

FIG. 4 is a schematic diagram illustrating liver spheroid formation in low-binding 96-well plates.

FIG. 5A demonstrates the formation of advanced liver organoids formed by 3-cell (hepatocyte, hepatic stellate cells, and liver sinusoidal endothelial cells) as well as 5-cell (hepatocyte, hepatic stellate cells, liver sinusoidal endothelial cells, cholangiocytes, and liver macrophages—Kupffer cells) in hepatocyte maintenance medium (HMM) or in 3C medium.

FIG. 5B demonstrates increased albumin expression in 5-cell organoids with 3C medium (3CM) on day 21.

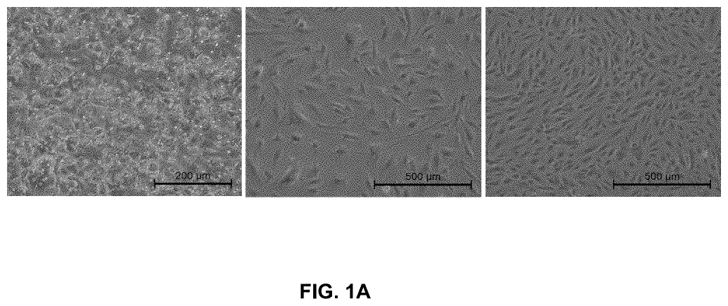

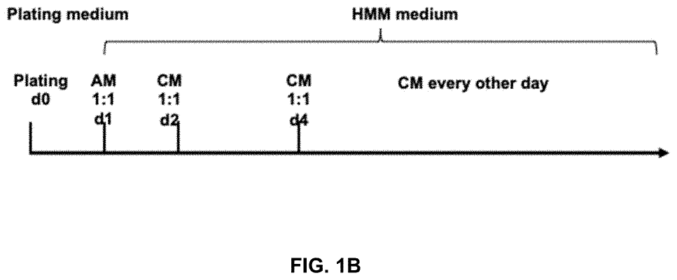

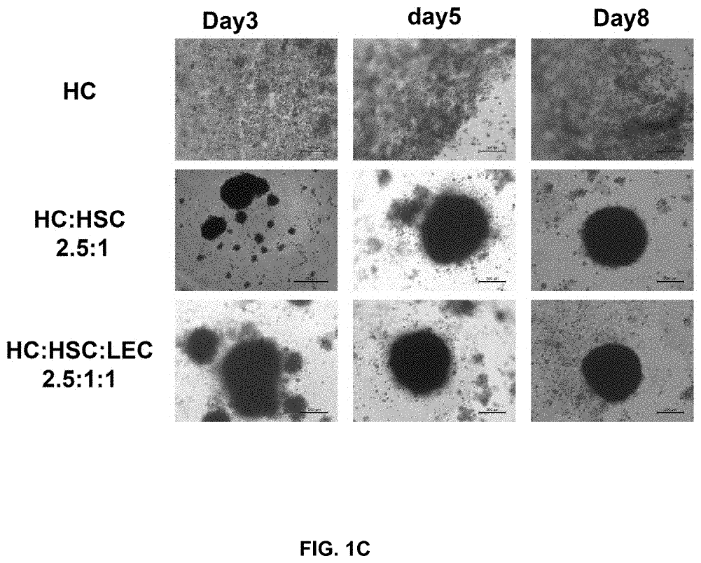

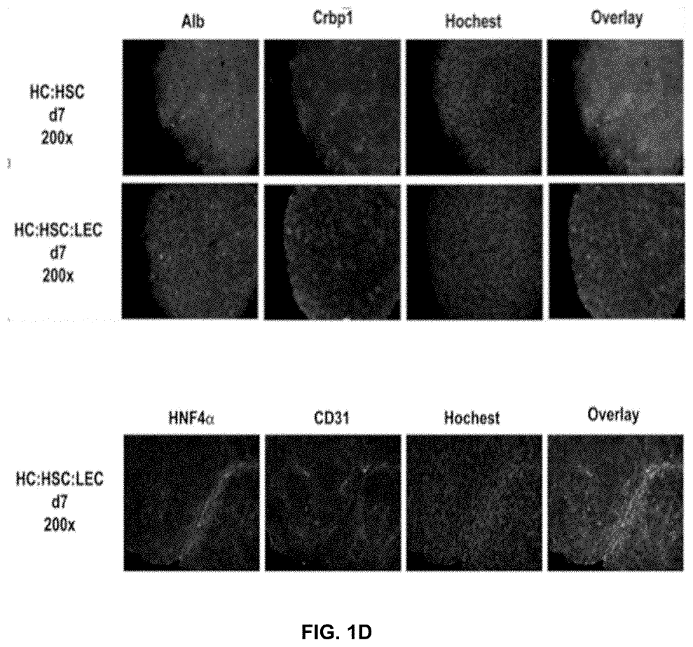

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

All publications, including but not limited to patents and patent applications, cited in this specification are herein incorporated by reference as though set forth in their entirety in the present application.

The methods and compositions provided herein are based at least in part on the inventors' development of three-dimensional spheroids that mimic liver tissue.

Accordingly, in a first aspect provided herein is a substantially spheroid liver (“hepatic”) organoid comprising human hepatocytes (HC), hepatic stellate cells (HSC) and liver sinusoidal endothelial cells (LSEC). As demonstrated herein, such hepatic organoids maintain functional hepatocytes for several weeks in culture as demonstrated by Albumin, CYP3A4, Sox9, HNF4a, and CK-19. As used herein, the term, “organoid” refers to an engineered material, produced in vitro, that comprises complex topologies and geometries that recapitulate some in vivo physiological conditions and cell-to-cell interactions found within native tissues. In exemplary embodiments, the liver organoid is substantially spheroid, meaning that it has a three-dimensional shape that is generally round, although perfect roundness is not required. As demonstrated by the inventors, the spheroid shape forms spontaneously when a plurality of human cell types are cultured together under low-adhesion conditions in a round-bottom culture plate.

Hepatic spheroids of this disclosure can be produced by any appropriate method of aggregating cultured cells. Preferably, cells (e.g., a mixture of hepatocytes, hepatic stellate cells, and liver sinusoidal endothelial cells) are cultured on low-adhesion or non-adhesive plates under conditions that promote spontaneous aggregation into spheroids. Spheroid size is determined, at least in part, by cell number and culture time. In some cases, about 10,000 to 50,000 cells (e.g., about 10,000, about 20,000, about 30,000, about 40,000, about 50,000) of each type are used to prepare spheroids have a diameter of approximately 100-500 μm. In some cases, a total of 35,000-40,000 cells are seeded on a non-adhesive culture plates under conditions that promote spontaneous aggregation into spheroids. In other cases, the number of cells of each type can be increased or decreased as necessary used to generate spheroids have a diameter of approximately 100-500 μm (e.g., about 100, 200, 300 400, 450, 500 μm, inclusive) in about two to about three days in culture.

In some cases, the method of producing a substantially spheroid liver organoid comprises culturing a plurality of human cells under low-adhesion culture conditions in the presence of a culture medium comprising a TGFβ receptor inhibitor, forskolin, and a Notch inhibitor, the plurality of human cells comprising two or more cell types selected from the group consisting of hepatocytes, human hepatic stellate cells, and human liver sinusoidal endothelial cells, wherein the plurality of human cells are cultured in the medium for about 3 days to about 7 days or more (e.g., about 3, 4, 5, 6, 7, or more days) until a substantially spheroid liver organoid is obtained. Preferably, the plurality of human cells are cultured in the medium for about 5 days to about 7 days (e.g., about 5,6,7 days, inclusive).

Hepatic spheroids comprising three liver cell types can be produced by culturing human hepatocytes (HC), hepatic stellate cells (HSC) and liver sinusoidal endothelial cells (LSEC) at a ratio of 2.5:1:1. It will be understood by those having ordinary skill in the art that ratios of cell types may vary based on the number of cell types, culture conditions, spheroid size, cell viability, and other variables.

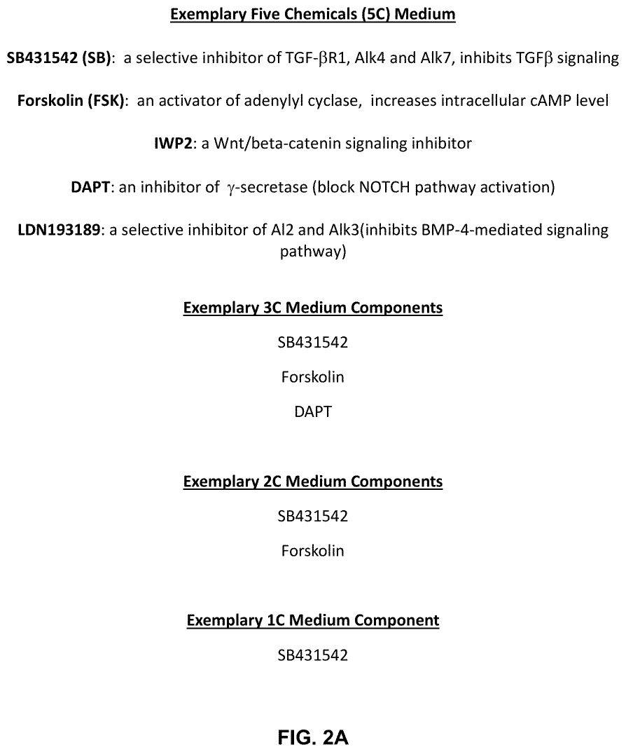

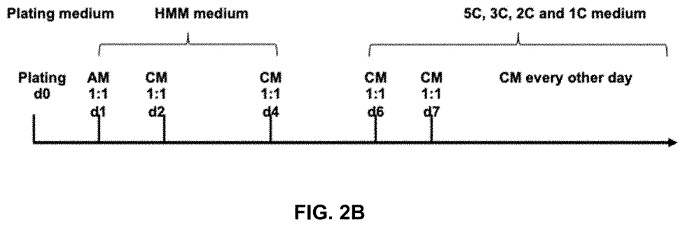

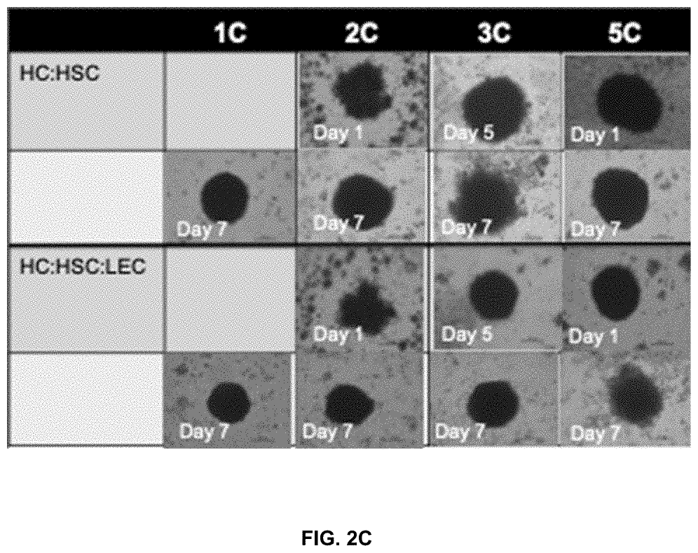

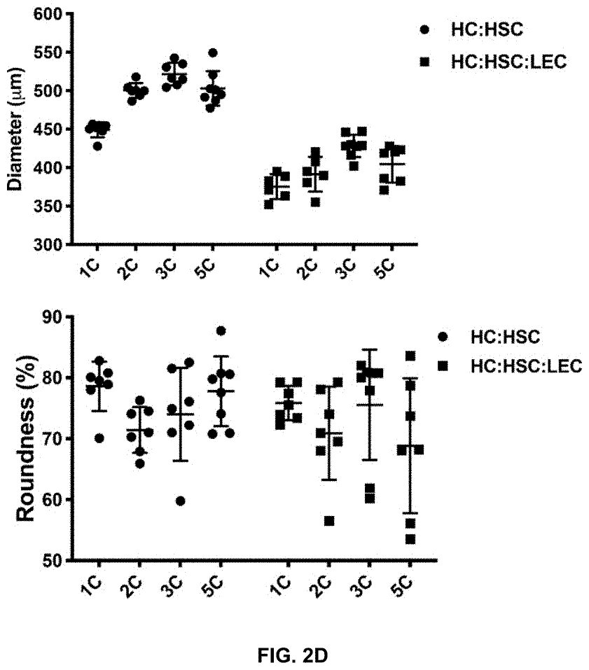

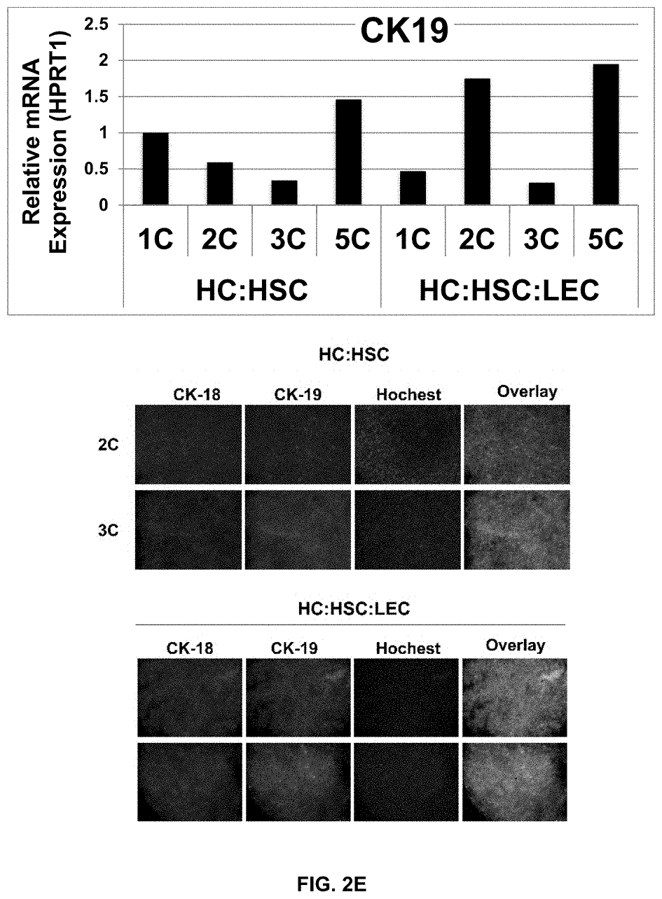

Referring to FIG. 2A, the culture medium preferably comprises of a TGFβ receptor inhibitor, activator of adenylyl cyclase, and a Notch pathway inhibitor. As used herein, the term “TGFβ receptor inhibitor” refers to a composition that directly inhibits TGFβ receptor, TGFβ receptor bioavailability, binding of TGFβ to TGFβ receptor, or the activation of Smad2/3 mediated signaling by TGFβRI. In some cases, the TGFβ receptor inhibitor is 4-[4(1,3-Benzodioxol-5-yl)-5-(2-pyridinyl)-1H-imidazol-2-yl]-benzamide (SB431542). Other TGFβ receptor inhibitors include, without limitation, 2-(5-Chloro-2-fluorophenyl)-4-[(4-pyridyl)amino]pteridine (SB208), 6-[2-(1,1-Dimethylethyl)-5-(6-methyl-2-pyridinyl)-1H-imidazol-4-yl]quinoxaline (SB525334), and 4-(2-(6-methylpyridin-2-yl)-5,6-dihydro-4H-pyrrolo[1,2-b]pyrazol-3-yl)quinoline-6-carboxamide (LY2157299). In some case, activator of adenylyl cyclase is 3R, 4aR, 5S, 6S, 6aS, 10S, 10aR, 10bS)-5-(Acetyloxy)-3-ethenyldodecahydro-6,10,10b-trihydroxy-3,4a, 7,7,10a-pentamethyl-1H-naphtho[2,1-b]pyran-1-one (forskolin) without limitation. In some cases, the Notch inhibitor is a γ-secretase inhibitor as (2S)—N-[(3,5-Difluorophenyl)acetyl]-L-alanyl-2-phenyl]glycine 1,1-dimethylethyl ester (DAPT). Other Notch inhibitors include, without limitation, a dibenzazepine (DBZ), and a benzodiazepine (DB). In some cases, the culture medium comprises fewer ingredients (e.g., 1C or 2C medium in FIG. 2A) or more ingredients (e.g., 5C medium in FIG. 2A). For instance, “5C medium” comprises a TGFβ receptor inhibitor, forskolin, a Notch inhibitor, a Wnt inhibitor such as IWP2, and an inhibitor of BMP receptor-mediated signaling (e.g., LDN193189). In some cases, 5C medium comprises SB431542 (10 μM), forskolin (20 μM), DAPT(5 μM), IWP2(0.5 μM), and LDN193189 (0.1 μM).

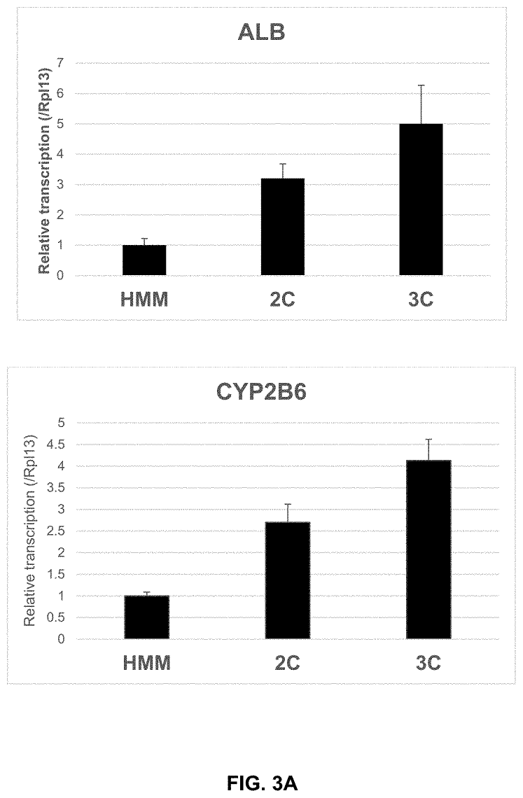

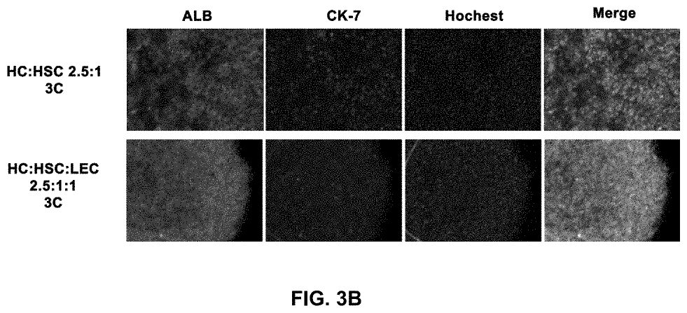

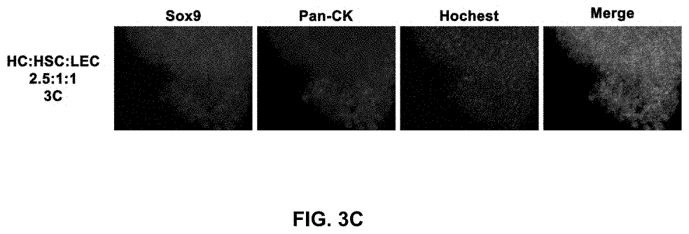

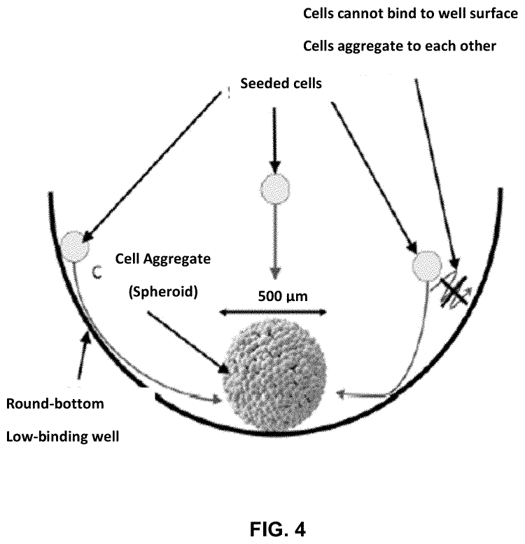

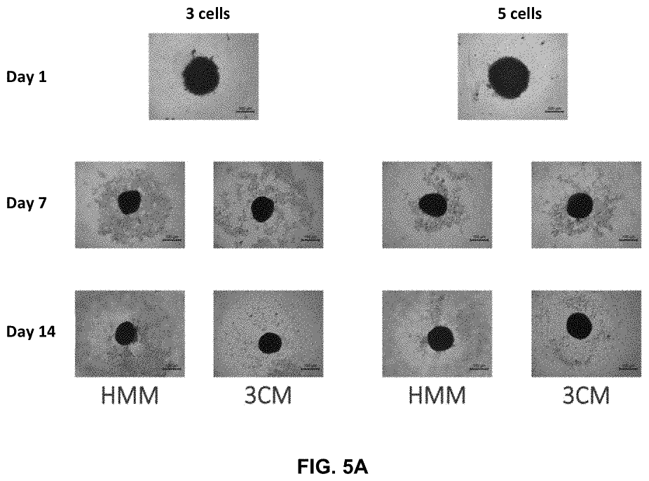

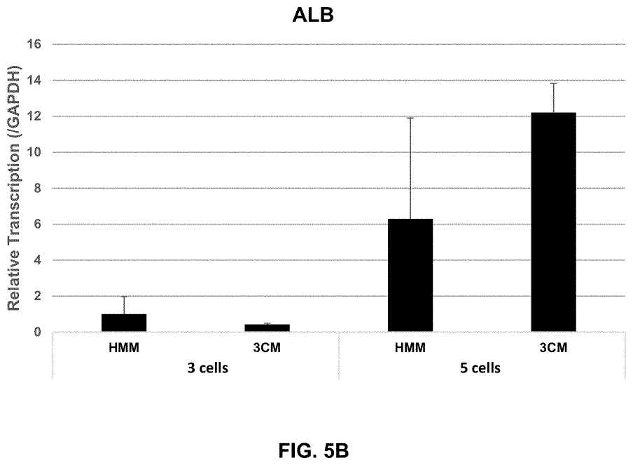

As shown in FIG. 2E, culturing in the presence of 5C medium condition can significantly upregulate CK19 expression in organoids comprising human hepatocytes (HC), hepatic stellate cells (HSC) and liver sinusoidal endothelial cells (LSEC) at a ratio of 2.5:1:1. In some cases, 3C medium comprises SB431542 (10 μM), forskolin (20 μM) and DAPT(5 μM). As shown in FIG. 3A, culturing in the presence of 3C medium condition can significantly upregulate ALBUMIN (ALB) and CYP3A4 expression in organoids comprising human hepatocytes (HC), hepatic stellate cells (HSC) and liver sinusoidal endothelial cells (LSEC) at a ratio of 2.5:1:1 when compared it to culturing in HMM (Lonza) or 2C medium (SB431542 and forskolin), which demonstrated that 3C medium best preserved mature hepatocyte function.

In some cases, cells or cell spheroids used to produce the synthetic tissue material are wild-type cells or may contain one or more synthetic or genetically engineered nucleic acids (e.g., a nucleic acid containing at least one artificially created insertion, deletion, inversion, or substitution relative to the sequence found in its naturally occurring counterpart). Cells comprising one or more synthetic or engineered nucleic acids are considered to be an engineered cell. As used herein, the terms “tissue” and “tissue construct” refer to aggregates of cells. In some cases, a substantially spheroid liver organoid produced according to the methods described herein may comprise recombinant or genetically-modified cells in place of or in addition to unmodified or wild-type (“normal”) cells. For example, it can be advantageous in some cases to include recombinant/genetically-modified cells that produce recombinant cell products, growth factors, hormones, peptides or proteins (e.g., detectable reporter proteins) for a continuous amount of time or as needed such as, for example, when biologically, chemically, metabolic or thermally signaled due to the conditions present in culture. Procedures for producing genetically modified cells are generally known in the art, and are described in Sambrook et al, Molecular Cloning, A Laboratory Manual, Cold Spring Harbor Press, Cold Spring Harbor, N.Y. (1989), incorporated herein by reference.

Cells for spheroids can be generated, harvested, and/or cultured according to any appropriate protocols. In some cases, cells for substantially spheroid liver organoids can be generated from enzymatically dissociated (e.g., trypsin treated) and/or mechanically dissociated tissues of interest, from cell lines, or from stem cells (e.g., directed differentiation of stem cells into a cell type of interest).

Although human cells are preferred for use in substantially spheroid liver organoids of this disclosure, it may be advantageous in some instances to prepare organoids comprising non-human cells. For example, it may be advantageous to use cells obtained from other mammalian species including, without limitation, equine, canine, porcine, bovine, feline, caprine, murine, and ovine species. Cell donors may vary in development and age.

Any appropriate method(s) can be used to assay substantially spheroid liver organoids for viability and tissue-specific metabolic activity. For example, substantially spheroid liver organoids can be tested for urea and albumin production, and CYP activity assay. Suitable methods for detecting the presence or absence of biological markers are well known in the art and include, without limitation, immunohistochemistry, qRT-PCR, RNA sequencing, and the like for evaluating gene expression at the RNA level. In some cases, methods such as immunohistochemistry are used to detect and identify cell types or biomolecules within a hepatic spheroid. For example, whole liver tissue constructs or portions thereof can be stained for specific differentiation markers by immunohistochemistry. In some cases, it will be advantageous to perform dual-label immunofluorescence to assess the relative expression of individual marker proteins or to detect multiple progenitor or differentiated cell types within a construct. Appropriate primary and secondary antibodies are known and available to those practicing in the art. In addition, microarray technology or nucleic acid sequencing (e.g., RNA sequencing) can be used to obtain gene expression profiles for hepatic spheroids. For instance, spheroids that maintain hepatocyte function can be determined based on production of urea and Albumin (Alb), glycogen storage, and expression of liver genes including, without limitation, α-fetoprotein (AFP), Alb, CYP3A4, Sox9, hepatocyte nuclear factor 4α (HNF4α), and cytokeratin 19 (CK-19). Quantitative methods for evaluating expression of markers at the protein level in cell populations are also known in the art. For example, flow cytometry is used to determine the fraction of cells in a given cell population that express or do not express biological markers of interest.

In another aspect, the present invention provides methods for producing and using substantially spheroid liver organoid for high throughput screening of candidate test compounds, e.g., for drug liver toxicity screening, drug screening, drug discovery, or drug response. In particular, provided herein are methods in which a hepatic spheroid obtained as described herein is used to screen test compounds for known and unknown toxicities and for efficacy in treating liver disease. For example, a hepatic spheroid comprising HC, HSC, and LSEC can be contacted to a test compound and assayed for any effect on any of the cell types contained therein (e.g., hepatocytes, hepatic stellate cells, LSECs). In certain embodiments, screening methods comprise contacting one or more test compounds to a hepatic spheroid obtained as described herein and detecting a positive or negative change in a biological property or activity such as, without limitation, gene expression, protein expression, cell viability, cell proliferation, inflammation and subsequent fibrosis. Advantages of the screening methods provided herein are multifold and include the ability to rapidly screen test substances for activity beneficial for treatment or prevention of liver disease without having to wait for liver disease to occur in a subject. Indeed, the in vitro screening methods can be conducted without the need for a human subject or animal models. The methods can be conducted economically (e.g., using multi-well plates that require minimal amounts of a test compound and relatively inexpensive hydrogel materials) and are readily adapted to high throughput methods (e.g., using robotic or other automated procedures).

As described herein, the screening methods of this disclosure comprise analysis of several induction strategies such as various dosing and durations of insults including, without limitation, various nutrients, inflammatory inducers, xenobiotics, liver fibrosis and metabolism. In addition, the methods encompass overlaying a range of modulatory approaches to profile prophylactic and treatment oriented drug strategies on human hepatocytes, human liver sinusoidal endothelial cells (LSECs), or human hepatic stellate cells of the organoid.

The manner in which a test compound has an effect on a particular biological activity of the constructs of the present invention will depend on the nature of the test compound, the composition of the hepatic spheroid and the particular biological activity being assayed. However, methods of this disclosure will generally include the steps of (a) culturing a hepatic spheroid with a test compound, (b) assaying a selected biological activity of one or more cell types within hepatic spheroid, and (c) comparing values determined in the assay to the values of the same assay performed using a hepatic spheroid having the same composition as the construct contacted by the test compound but cultured in the absence of the test compound (or in the presence of a control). Detecting a positive or negative change in a biological property or activity of a cell of the hepatic spheroid can comprise detecting at least one effect of a test compound on morphology or life span of a cell within the contacted liver tissue construct, whereby a test compound that reduces the life span of the cells or the liver tissue as a whole, or has a negative impact on the morphology of the cells or the liver tissue, is identified as toxic to that cell or tissue. In some cases, detecting comprises performing a method such as RNA sequencing, gene expression profiling, transcriptome analysis, metabolome analysis, detecting reporter or sensor, protein expression profiling, Förster resonance energy transfer (FRET), metabolic profiling, and microdialysis. Test compounds can be screened for effects on gene expression in the contacted hepatic spheroid, where differential gene expression as compared to an uncontacted hepatic spheroid is detected.

In some cases, detecting and/or measuring a positive or negative change in a level of expression of at least one gene following exposure (e.g., contacting) of a hepatic spheroid to a test compound comprises whole transcriptome analysis using, for example, RNA sequencing. In such cases, gene expression is calculated using, for example, data processing software programs such as Light Cycle, RSEM (RNA-seq by Expectation-Maximization), Excel, and Prism. See Stewart et al., PLoS Comput. Biol. 9:e1002936 (2013). Where appropriate, statistical comparisons can be made using ANOVA analyses, analysis of variance with Bonferroni correction, or two-tailed Student's t-test, where values are determined to be significant at P<0.05. Any appropriate method can be used to isolate RNA or protein from a hepatic spheroid. For example, total RNA can be isolated and reverse transcribed to obtain cDNA for sequencing.

As used herein, “test compounds” are not particularly limited and include, for example, single compounds such as natural compounds, organic compounds, inorganic compounds, proteins, antibodies, peptides, and amino acids, as well as compound libraries, expression products of gene libraries, cell extracts, cell culture supernatants, products of fermenting microorganisms, extracts of marine organisms, plant extracts, prokaryotic cell extracts, unicellular eukaryote extracts, and animal cell extracts. These may be purified products or crude purified products such as extracts of plants, animals, and microorganisms. Also, methods for producing test compounds are not particularly limited; test substances may be isolated from natural materials, synthesized chemically or biochemically, or prepared by genetic engineering. “Test compounds” also encompass mixtures of the above-mentioned substances.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

1. A substantially spheroid liver organoid comprising two or more cell types selected from the group consisting of hepatocytes, human hepatic stellate cells, and human liver sinusoidal endothelial cells.

2. The organoid of claim 1, wherein the two or more cell types are human hepatocytes (HCs), human hepatic stellate cells (HSCs), and human liver sinusoidal endothelial cells (LSECs).

3. The organoid of claim 2, wherein the organoid exhibits hepatic function for at least 2 weeks in culture.

4. The organoid of claim 2, wherein the organoid exhibits hepatic function for about 3 weeks in culture.

5. A method of producing a substantially spheroid liver organoid, comprising culturing a plurality of human cells under low-adhesion culture conditions in the presence of a culture medium comprising a TGFβ receptor inhibitor, an activator of adenylyl cyclase, and a Notch inhibitor, the plurality of human cells comprising two or more cell types selected from the group consisting of hepatocytes, human hepatic stellate cells, and human liver sinusoidal endothelial cells, wherein the plurality of human cells are cultured in the medium for about 3 days to about 7 days until a substantially spheroid liver organoid is obtained.

6. The method of claim 5, wherein the two or more cell types are human hepatocytes (HCs), human hepatic stellate cells (HSCs), and human liver sinusoidal endothelial cells (LSECs).

7. The method of claim 5, wherein the substantially spheroid liver organoid exhibits hepatic function for at least 2 weeks in culture.

8. The method of claim 5, wherein the substantially spheroid liver organoid exhibits hepatic function for about 21 days in culture.

9. The method of claim 5, wherein the plurality of human cells comprises HCs, HSCs, and LSECs at a ratio of 2.5:1:1.

10. The method of claim 5, wherein the culture medium comprises a TGFβ receptor inhibitor, an activator of adenylyl cyclase, and a Notch inhibitor.

11. The method of claim 10, wherein the TGFβ receptor inhibitor is SB431542.

12. The medium of claim 10, wherein the Notch inhibitor is DAPT.

13. The method of claim 10, further comprising an inhibitor of Wnt signaling and an inhibitor of BMP signaling.

14. The method of claim 13, wherein the medium comprises SB431542, forskolin, DAPT, IWP2, and LDN193189.

15. A substantially spheroid liver organoid obtained according to the method of claim 5.

16. A method of testing a compound, the method comprising
contacting the test compound to a substantially spheroid liver organoid comprising human hepatocytes, human liver sinusoidal endothelial cells (LSECs), and human hepatic stellate cells; and
detecting the effect of the compound on one or more cell types within the substantially spheroid liver organoid.

17. The method of claim 16, wherein the substantially spheroid liver organoid is obtained according to the method of claim 5.

18. The method of claim 16, wherein the test compound exerts a therapeutic effect on human hepatocytes, human liver sinusoidal endothelial cells (LSECs), or human hepatic stellate cells of the organoid.
